(19) United States
(12) Patent Application Publication
(10) Pub. No.: US 2019/0380600 A1
(43) Pub. Date: Dec. 19, 2019

(54) Electronic Biometric Devices and Methods of Construction

(71) Applicant: DST Innovations Limited, Bridgend (GB)

(72) Inventor: Anthony Miles, Bridgend (GB)

(73) Assignee: DST Innovations Limited, Bridgend (GB)

(21) Appl. No.: 16/341,662

(22) PCT Filed: Oct. 9, 2017
(86) PCT No.: PCT/GB2017/053053

§ 371 Date: Apr. 12, 2019

(30) Foreign Application Priority Data
Oct. 12, 2016 (GB) 1617310.6

(51) Int. Cl. A61B 5/024; A61B 5/00

(52) U.S. Cl. CPC A61B 5/02438; A61B 5/6833; H05K 3/321; A61B 5/681; A61B 5/684

(57) ABSTRACT

A biometric sensor device for attachment to the skin of a user comprises an electronic device arranged to receive biometric signals or data from the user and a power cell connected or connectable to the electronic device for providing power thereto, the electronic device and the power cell being provided as discrete, thin, flexible parts connectable or connected together. Methods of construction of the device and applications for use of the device are also disclosed.

### FIELD OF THE INVENTION

The present invention relates to electronic biometric devices, as well as methods of construction and applications thereof.

### BACKGROUND OF THE INVENTION

There are many types of biometric devices available today, ranging from smart watches and wristbands to arm bands, smart shoes, smart clothes and smartphones that we carry around in our pocket. Many of these devices are expensive, bulky and don't give very accurate or useful data. Part of the problem is that they are designed to be strapped to your wrist, strapped to your chest, built into something that you wear, or sometimes pressed to your finger. This means that the device reading the biometric data is located in a place that can make it uncomfortable to wear and isn't necessarily in a good location for the collection of that data. Current devices are designed to be used one at a time and in many cases, the collection of the data is not the only function they perform. It is often cost prohibitive to buy more than one of the currently available devices, and in many situations, they are not compatible with some smartphones or computers, so the consumer is often limited to a specific device and its platform, along with its associated restrictions.

Some devices have started to enter into the market that are designed to address the aforementioned problems; however they still use conventional electronics encased in a plastic casing. This electronic device is then connected to a patch that provides adhesion to the user's skin. Such a device is described in WO 2014/165071 A1.

### STATEMENTS OF THE INVENTION

Aspects of the present invention are defined by the accompanying claims.

Embodiments of a device and its energy cell according to the present invention are of novel design and construction enabling them to be thinner, lighter, and much more cost effective to produce.

Embodiments of the present invention aim to address some of the problems inherent with the aforementioned devices and systems. Embodiments may include a method of construction that substantially reduces the overall device cost to the end user, and provides a platform for the development of uses, software, firmware and connectivity, in areas such as, but not limited to, medical applications, health and fitness applications, sports activity monitoring applications, military applications, and crowd movement applications.

An embodiment of the invention comprises an extremely thin, printed electronic, lightweight and energy efficient multi-sensor device that is designed to be placed in direct contact with the surface of the skin. The location of the device will depend on the type of biometric data to be collected. In the case where multiple data points are needed, more than one device can be placed in multiple locations on the body and wirelessly connected to provide a more detailed data set of the subject's activities, both recorded and in real time.

The biometric data can be associated together to describe a group of people, giving a direct real-time analysis of the group. This can be useful when a comparison of the individuals against a known data set is needed or in the case of a number of individuals using the device, such as a team, to compare the performance against each other and/or historical data sets. The data that is collected may include for example heart rate, temperature, perspiration composition, position of the device on the body, body orientation, movement, body impact and/or geographical location. With additional accessories the device(s) may also measure, for example, joint movement, pressure points on the body and/or respiration.

### BRIEF DESCRIPTION OF THE DRAWINGS

Specific embodiments of the present invention will now be described with reference to the following drawings.

FIG. 1 shows schematically the architecture of a device and power cell in a first embodiment of the invention.

FIG. 2 shows a front view of the device.

FIG. 3 shows a back view of the device.

FIG. 4 shows a side view of the device.

FIG. 5 shows a power cell for use with the device.

FIG. 6 shows the back of the power cell.

FIG. 7 shows a side view of the power cell

FIG. 8 shows the device being placed over the top of the power cell.

FIG. 9 shows a side view of the device and power cell being placed together.

FIG. 10 shows a side view of the device and the power cell together and ready to use.

FIGS. 11 to 19 show steps of construction of the device.

FIG. 20 shows schematically the architecture of a device and power cell in a second embodiment of the invention.

FIG. 21 shows a front view of the device.

FIG. 22 shows a back view of the device.

FIG. 23 shows a side view of the device.

FIG. 24 shows a front view of the power cell for use with the device.

FIG. 25 shows a back view of the power cell.

FIG. 26 shows a side view of the power cell.

FIG. 27 shows the power cell being placed on the device.

FIG. 28 shows schematically the components of the electronic circuitry of the device in the first or second embodiments.

FIG. 29 shows a display of the first part of a connection setup to a device in an embodiment.

FIG. 30 shows a series of displays for selecting an orientation of a body graphic.

FIG. 31 shows a series of displays for selecting the location of a device.

FIG. 32 shows a display for selection of the biometric data to be monitored.

FIG. 33 shows configuration screens for selecting sharing options for the biometric data.

FIG. 34 shows displays of searching for, and connecting to a device.

FIG. 35 illustrates a Bluetooth® connection between a smartphone and the device.

FIG. 36 illustrates a possible communication scenario for use with embodiments of the invention.

### DETAILED DESCRIPTION OF EMBODIMENTS

Specific embodiments the invention will now be described purely by way of example, and without restriction of the scope of aspects of the invention.

### First Embodiment

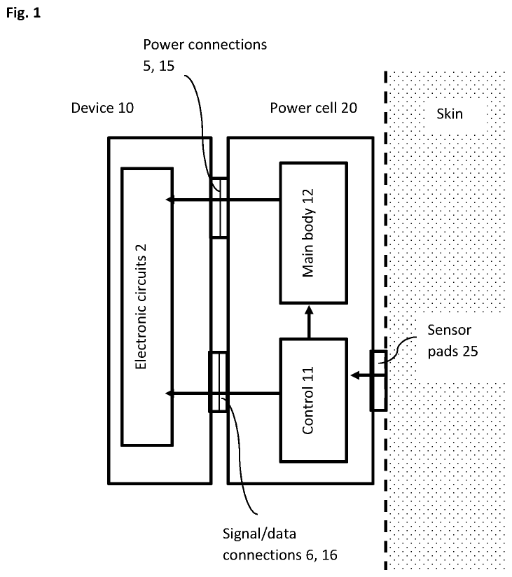

In a first embodiment, as shown in FIG. 1, a device 10 is connected to a power cell 20 through respective power connections 5, 15 and signal/data connections 6, 16. The power cell 20 comprises a main body 12 for supplying electrical energy to the device 10 via power connections 5, 15 and an electronic control 11 for controlling the supply of energy. The power cell 20 includes sensor pads 25 which contact the skin of the user. Signals/data sensed from the skin of the user are passed to the device 10 through signal/data connections 6, 16. The sensed signals may be processed by the electronic control 11 before being output to the device 10.

Structure

FIG. 2 shows a front view of the device 10 in a first embodiment, comprising: an encapsulating, soft and flexible pad 1 that houses and protects electronic components needed to provide the functionality of the device; a soft, flexible and thin electronic printed circuit 2 that is designed to connect the device 10 to a power cell 20 and to subsequently connect to electrodes that contact the body; an arrow 3 or other indicium that indicates alignment of the device to the power cell; and outer edge 4 that is designed in such a way as to provide excellent adhesion of the device to the power cell 20, ensuring a waterproof barrier.

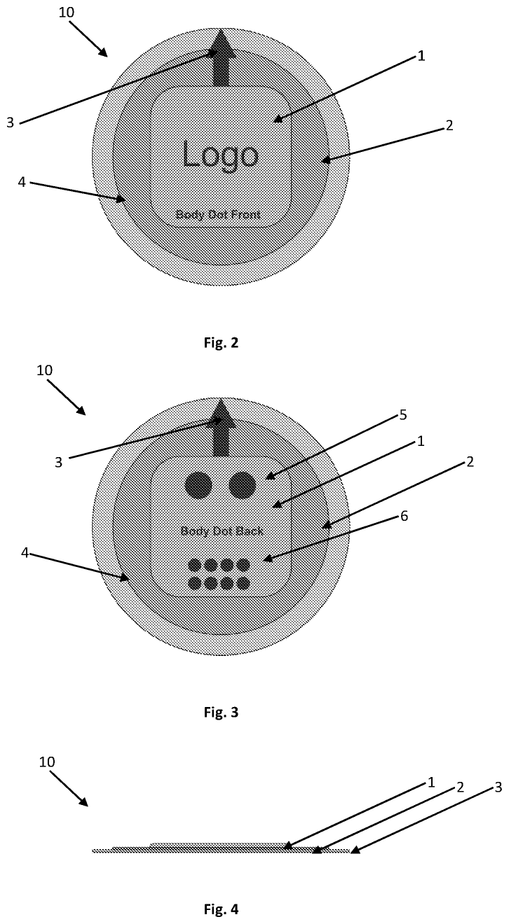

As shown in FIG. 3, the back of the device 10 includes connection pads 5 that connect the positive and negative terminals of the power cell 20 to the device 10 to power the electronic printed circuit 2, and data connection pads 6 to connect to corresponding pads in the power cell to carry biometric signals, derived from pads that are in direct contact with the user's skin. These signals will then be sent to the electronic printed circuit 2, to provide the functionality of the device 10.

FIG. 4 shows the flexible pad 1, the electronic printed circuit 2 and the outer edge 4 of the device 10, provided as layers.

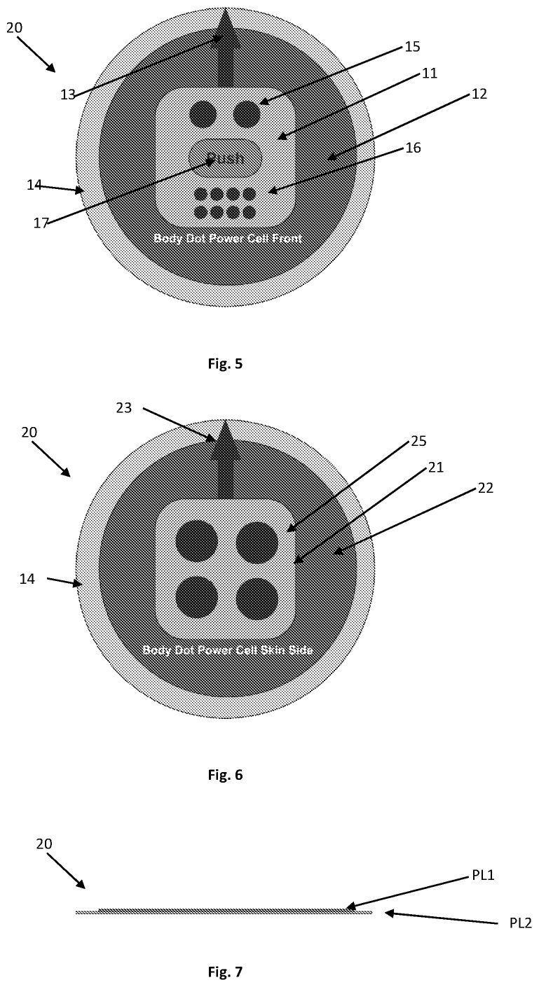

FIG. 5 show the front/upper side of the power cell 20, comprising: electronic control 11 that manages the power cell 20 and its connection to the device 10; main body 12 containing the energy producing chemistry, as known in the art; alignment arrow 13 that enables the power cell 20 and the device 10 to be correctly aligned and connected; water resistant edge 14; positive and negative contacts 15 that connect the power from the power cell to the device; data connection pads 16 that connect to corresponding pads in the device 10 to carry information from the sensor pads 25 that are in direct contact with the user's skin; and button 17 that, when pressed, activates the power cell 20.

FIG. 6 shows the back/skin side of the power cell 20, comprising: soft padded area 21 that holds skin contact electronics; protective layer 22 that ensures no contact between the power cell internal structure and the user's skin; contact alignment arrow 23; water resistant edge 14 and sensor pads 25 that will be in direct contact with the user's skin.

FIG. 7 shows a side view of the power cell, comprising: a first layer PL1 containing the electronic circuit 11, main body 12, alignment arrow 13, contacts 15 and data connection pads; and a second layer PL1 including the water-resistant edge 24 and sensor pads 25.

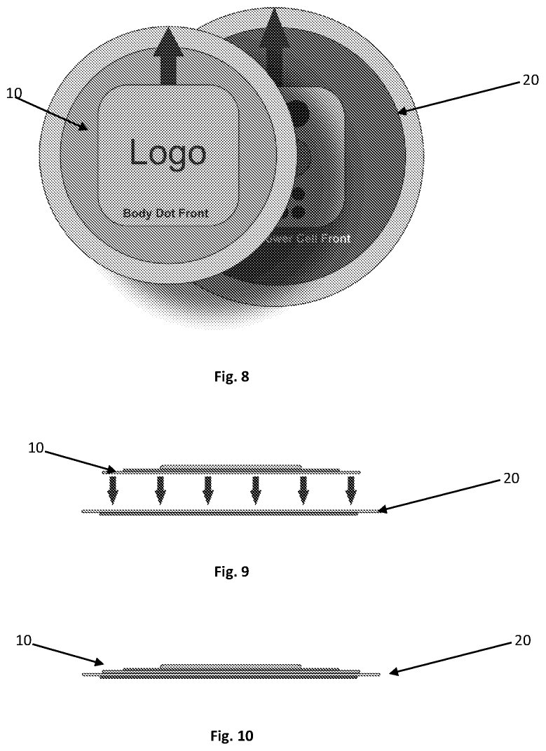

As shown in FIGS. 8-10, the device 10 is connected to the power cell 20 as follows. An adhesive protector is removed from the front/upper side of the power cell 20 and the device 10 is placed on top of the power cell 20, with the arrows 3 and 13 aligned. The device 10 is then activated, for example by pressing a button to release electrolyte in the power cell 20.

The user then removes a second adhesive protector from the back/skin side of the power cell 20, exposing the sensor pads 25. The connected device 10 and power cell 20 are then ready to place in the desired location on the body.

Device Construction

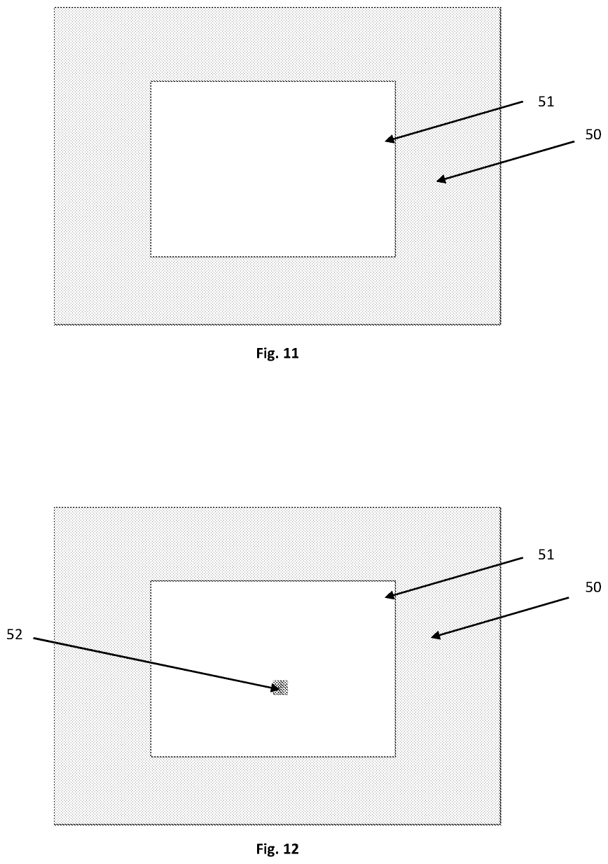

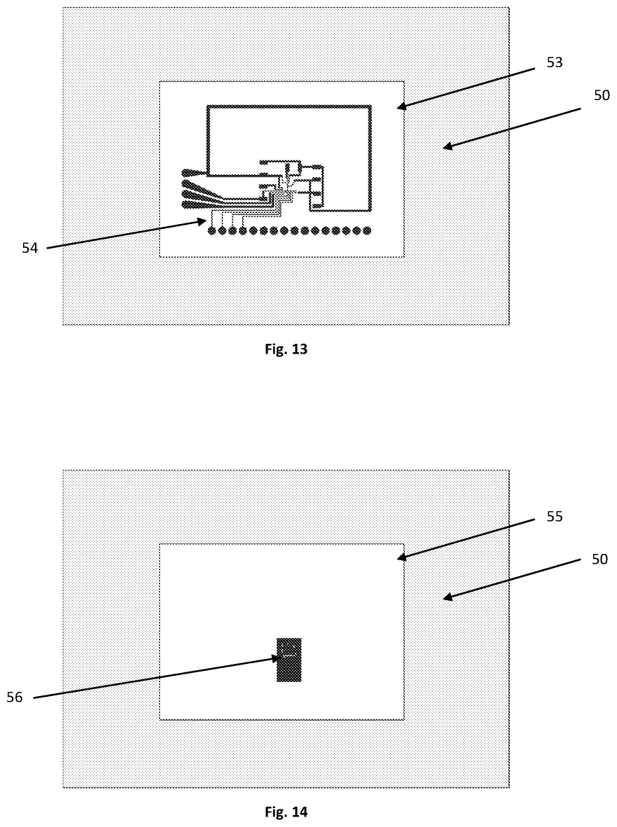

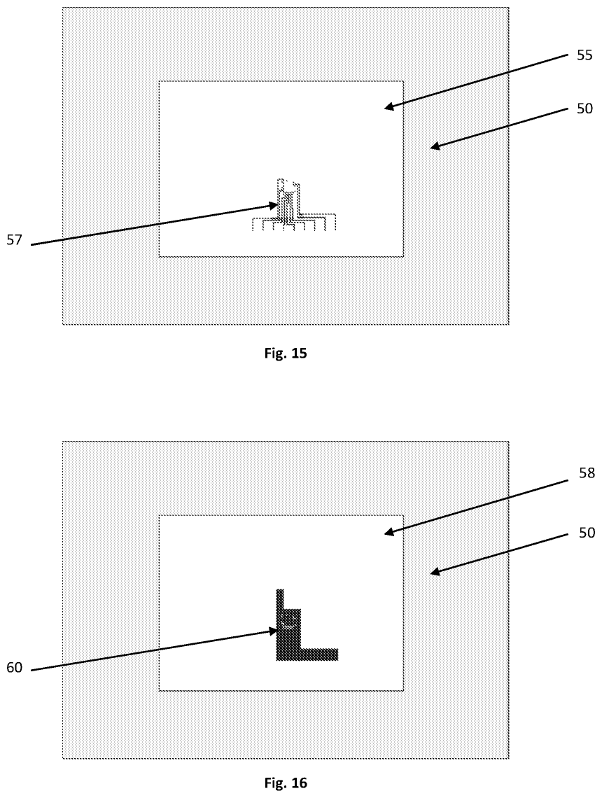

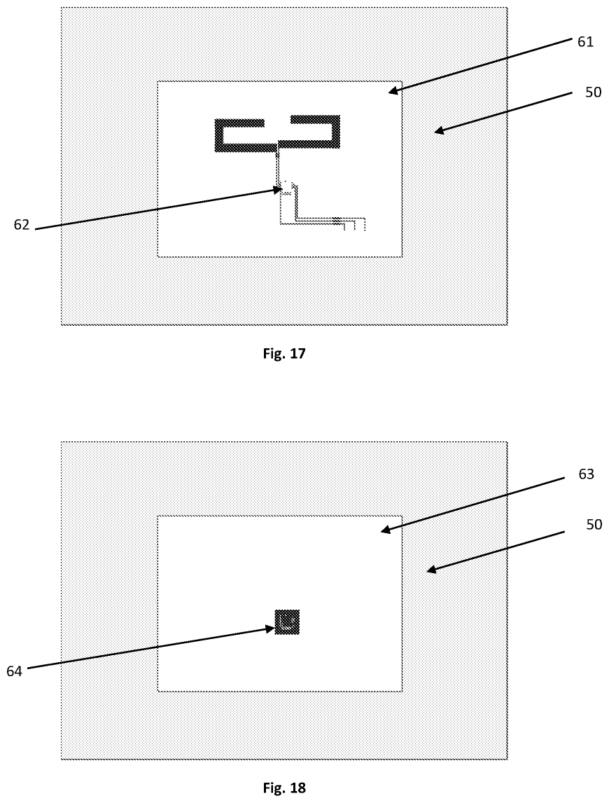

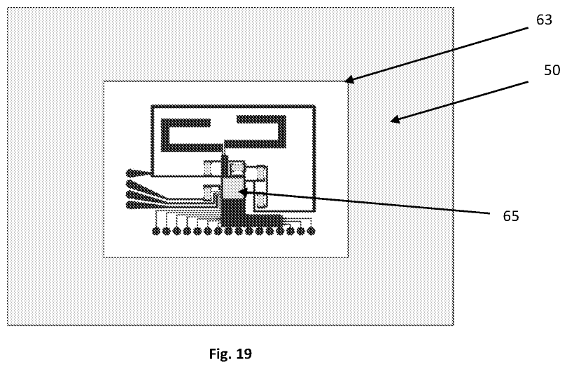

A method of construction of the device 10 will now be described, with reference to FIGS. 11 to 19 and to the following steps, which are preferably carried out in sequential order.
1. Pre-prepare a release liner 50 to ensure that it is free of contaminants. The release liner provides a stable material for the curing process. The release liner 50 may be pre-heated to remove any moisture and prevent shrinking during the process. The temperature of the pre-treatment will depend on the properties of the material being used as the release liner 50.
2. Print on to the release liner the adhesive layers which will stick the device 10 to the power cell 20. The printed adhesive layer may comprise three different types of adhesive. The first, inner adhesive is used to provide a semi-permanent bond between the device 10 and the power cell 20. The second adhesive is printed where the electrical contacts will be, and is designed to provide good adhesion to the terminals and ensure good electrical contact. The third adhesive is provided around the edge of the device 10 to ensure a good seal that is hydrophobic and prevents moisture from getting between the device 10 and the power cell 20.
3. A first layer of printable substrate 51 is then printed over the top of the adhesive layers, as shown in FIG. 11. This layer has a number of holes in it that correspond to the printed adhesive pads that promote the electrical contact and connection stability between the power cell 20 and the device 10.
4. A layer of conductive ink is printed in the holes to make the first layer of electrical contacts 52, as shown in FIG. 12.
5. A layer of soft and flexible printable substrate 53 is printed so as to make the holes on the prior layer smaller and to provide more structural integrity to the device 10.
6. A first layer 54 of the printed circuit 2 is printed using one or more conductive inks, as shown in FIG. 13.
7. A layer of insulator 55 is printed over the top of the first layer of printed circuit 2 so that this first layer of printed circuit 2 can be separated from the next layer. In areas where the first layer of printed circuit board and the second layer of printed circuit need to be connected, a number of suitably placed holes are formed in the insulator.
8. The holes that have been formed in the insulator are filled with a conductive material 56, as shown in FIG. 14. This conductive material forms a connection between the layers of the printed circuit 2, similar to a via in a standard printed circuit board structure.
9. A second layer 57 of printed circuit 2 is printed over the top of the preceding layers, as shown in FIG. 15.
10. This process is repeated until the required multi-layered connection system is complete. For example, as shown in FIGS. 16 to 18, a second layer of insulator 58 is printed over the top of the preceding layers, with holes that are filled with conductive material 60 for providing connections to the second layer 57 of printed circuit. A third layer 62 of printed circuit may then be printed over the second insulator 57, with connections to second layer 57 through the conductive material 60. A third layer of insulator 63 is then printed over the top of the preceding layers, with holes that are filled with conductive material 64 for providing connections to the third layer 62 of printed circuit.
11. After all the layers of the conductive circuits are printed, a microprocessor 65 and/or other components such as capacitors, diodes and resistors are added. The components may be connected to the conductive material 64 using pressure bonding, for example with a conductive adhesive. FIG. 19 shows the overall configuration of all of the layers of printed circuit.
12. A layer of insulating adhesive printed in specific support shapes is added and partly cured. Then a conductive adhesive is added that will hold the contacts in place when they are added to the printed multi-layer circuits using industry standard methods.
13. Once the components have been added, a soft gel layer is printed over them to protect them from being damaged, to form the pad 1. Then a logo and other information may be added.
14. The completed assembly may now be removed from the release liner 50 and bonded to other layers to form the device 10, or the other layers may be printed before removal from the release liner.

In some cases, the adhesives that are added in step 2 may be added as a last process once the device has been removed from the release liner. Also, in some cases, the soft gel protective layer may be printed on a separate release liner and added to the device at the assembly and packaging stage. The device 10 may be removed from the release liner(s) before packaging, or immediately before use.

Power Cell Structure

The power cell 20 is very similar in construction to the device 10 with the exception that the power cell 20 is printed in two halves which are then joined together using a pressure adhesive. In the case of a primary power cell 20, a capsule containing the electrolytes is placed between the two layers in such a way as to make it breakable. This releases the content activating the reaction. The chemistry used to construct the internal layers of the energy cell are known in the art, however proprietary formulations may be used is specific cases.

### Second Embodiment

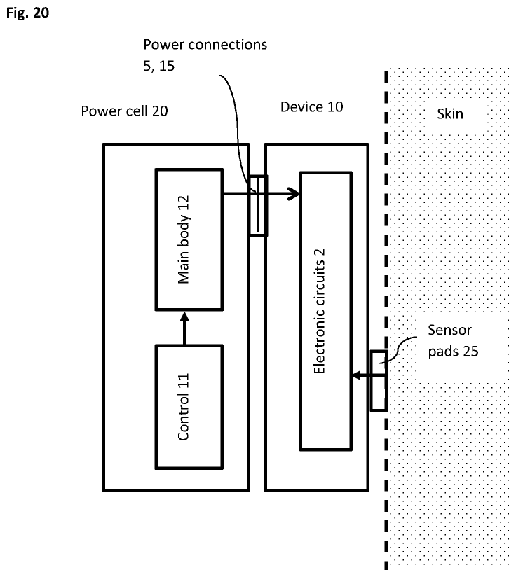

A second embodiment, as shown in FIG. 20, differs from the first embodiment in that the device 10 is designed to be placed in contact with the user's skin, and the power cell 20 is connected to the outer face of the device 10.

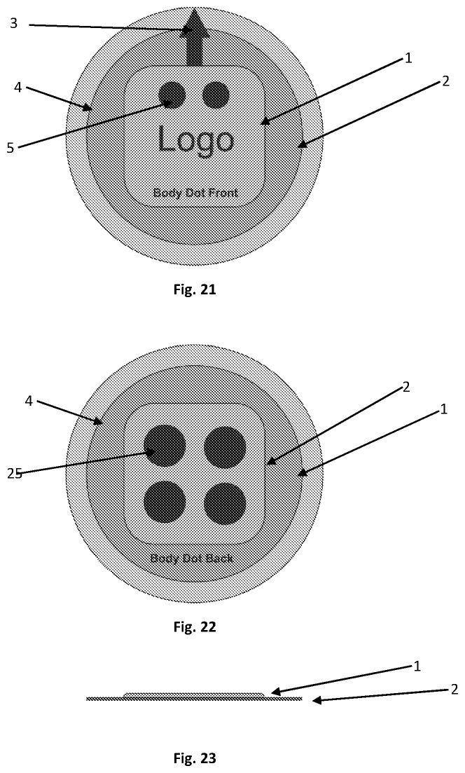

FIG. 21 shows a front view of a second embodiment of the device 10, comprising an encapsulating soft and flexible pad 1 that houses and protects the electronic components needed to provide the functionality of the device 10; soft, flexible and thin electronic printed circuit 2 that is designed to connect the device 10 to a power cell and subsequently connect the electrodes that contact the body; arrow 3 that indicates alignment of the device 10 to the power cell 20; outer edge 4 that is designed in such a way as to provide excellent adhesion of the device 10 to the power cell 20 ensuring a waterproof barrier; and positive and negative connections 5 that carry power to the electronic components that provide the functionality of the device 10.

FIG. 22 shows a back view of the device 10, including sensor pads 25 that are designed to be placed directly on the skin of the user.

FIG. 23 shows a side view of the device 10, including the soft protective layer 1 that protects the electronics that are needed for the device's functionality, and flexible area that includes the flexible circuitry 2.

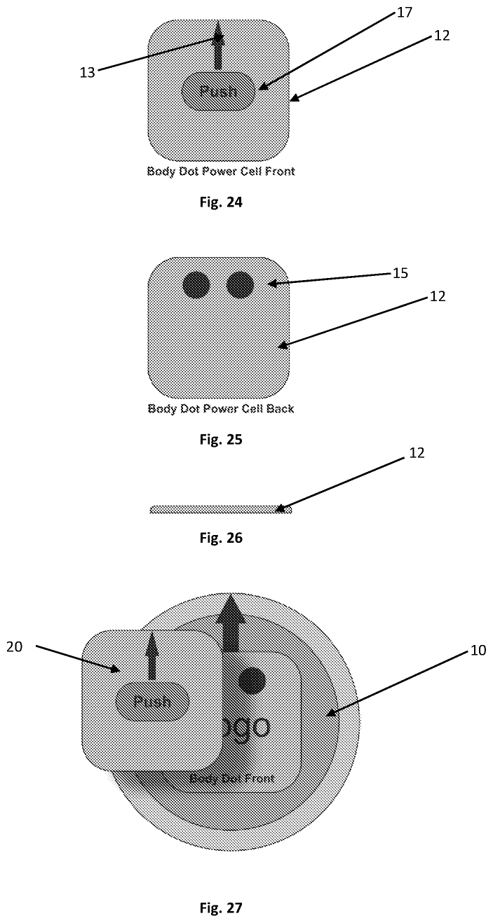

FIG. 24 shows a front view of a power cell 20 of the second embodiment, comprising soft and flexible casing 12 that contains the chemistry that enables the energy cell to, in the case of a primary cell, create the energy needed, and in the case of a secondary cell, retain the energy needed to power the device 10 and sustain its functionality for the desired time; and button 17 that in the case of a primary energy cell, will activate the chemical reaction.

FIG. 25 shows a back view of the power cell, including the positive and negative connection pads 15 that send the power needed to the device 10.

FIG. 26 shows a side view of the power cell 20.

FIG. 27 shows the power cell 20 being placed on the device 10. In this embodiment, the user will remove an adhesive protector from the back of the power cell 20 and place the power cell 20 on the front of the device 10. Once the device 10 is powered and activated, the user will then remove the adhesive protector layer from the back of the device 10 and place it in a location on their body.

Electronic Components of the Device

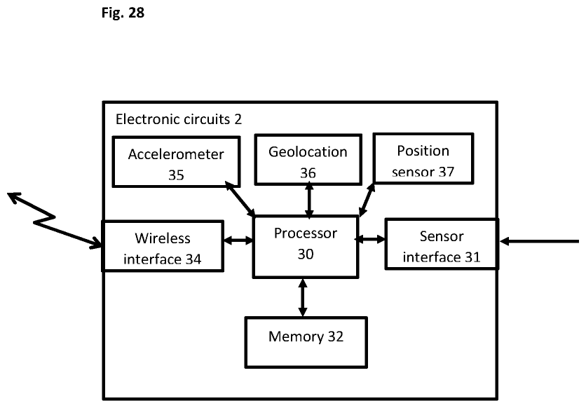

The device 10 of the first, second or other embodiments includes electronic circuits 2, which may include one or more of the following components, as shown for example in FIG. 28:
a processor 30 for processing the signals/data and/or controlling the device 10;
a sensor interface 31 for receiving signals/data sensed from the user's skin;
internal memory 32 for storing the sensed signals/data, other data received by the device 10, configuration data or settings for the device, and/or program code for execution by the processor;
one or more wireless communication interfaces 34, for example implementing Bluetooth®, Wi-Fi® and/or other standards;
an accelerometer 35 for detecting or measuring acceleration of the device 10;
geolocation circuitry 36, such a GPS and/or Glonass receiver, or beacon signal receiver; and
an orientation and/or position sensor 37 for sensing the orientation (e.g. with respect to the local magnetic field) and/or the absolute or relative position of the device 10.

### Connected Application Embodiments

The following describes an embodiment of a mobile phone application that may be used to connect to the device 10. Although this example is for a mobile phone application, alternative devices such as a tablet, computer, smart watch, or connected device could be used to utilise the functions of the device and visualise or present them in some way for the user. Visualisations or presentations may comprise one or more lights, displays, sounds, haptic feedback, or any combination thereof.

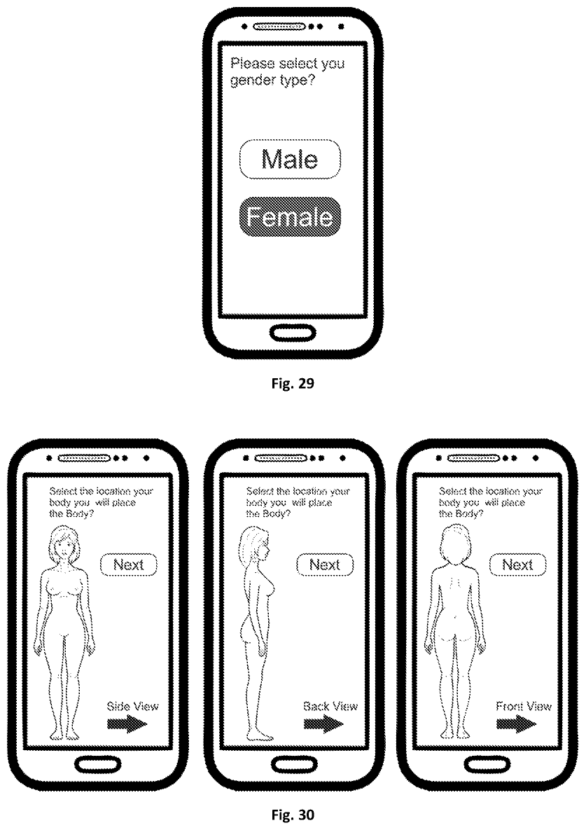

FIG. 29 shows the first part of the connection setup. It is preferable to establish the gender of the user of the device 10. The selection of Male or Female will set the device 10 and/or application into the correct mode. Alternatively, the gender may be determined automatically from information available to the application, or by sensing using the device 10.

The application then displays a graphic of a body of the determined gender, in this case female. The user can then select the location of a first device to be placed on their body. A first selection stage is to select the orientation of the body e.g. front, side or back, as shown in FIG. 30.

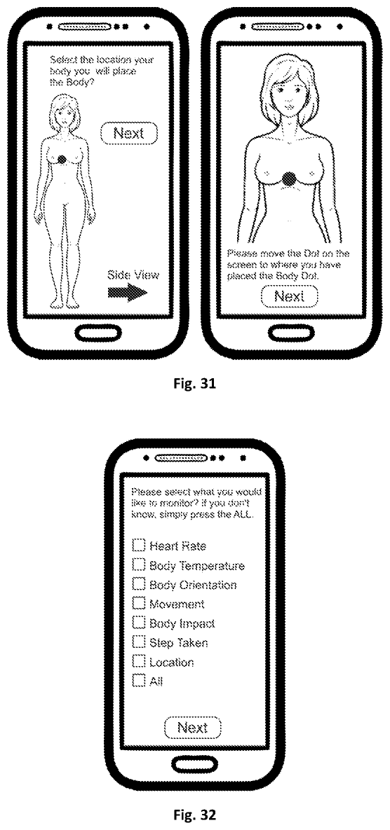

FIG. 31 shows that the front orientation has been selected. Once the front orientation is selected, the user is then prompted to make a location selection. Selecting a point on the graphic representation of the body will enlarge the graphic in the area so that the location of the device 10 can be more accurately determined.

FIG. 32 shows the next step of the display in which the location of the device has been determined, and the application and/or device 10 now needs to know what it should be monitoring, for example heart rate, body temperature, body orientation, movement, body impact, steps taken, and location. The user can select individual items from the list or simply select all.

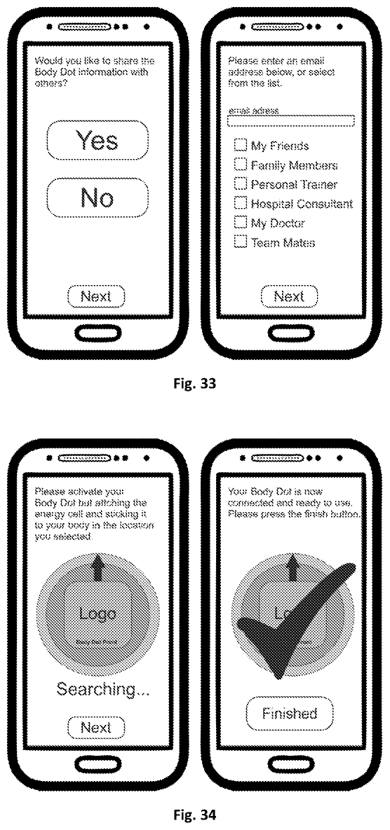

Now that the device 10 is connected to the mobile phone, the information collected by the device 10 can be communicated to other devices and assessable systems by any suitable means, such as email, text, social networks, websites, databases and other connected services. FIG. 33 shows possible configuration screens in a smartphone app that asks the user if they want to share the information and if they do, who they would like to share the information with and how they would like to share it.

FIG. 34 shows the smartphone app searching for the device 10 and confirming that it can access the information that is stored in internal memory of the device 10.

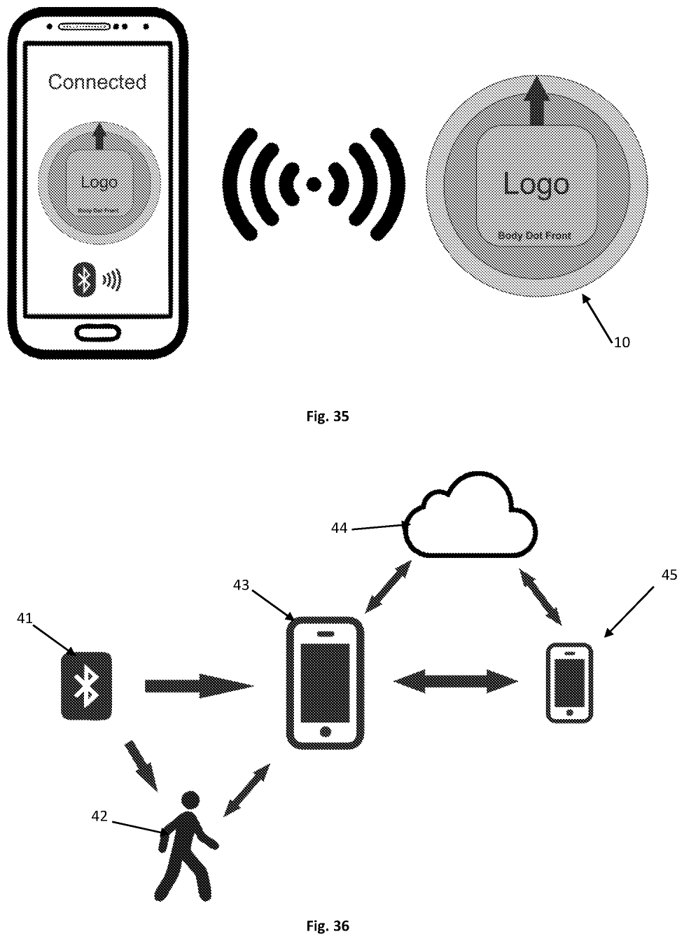

Once the device 10 is connected to a smartphone, tablet, computer, or other connected devices, information can be transmitted from the device 10 to the connected device and from the connected device to the device 10. Also, information can be sent to the connected device, such as a smartphone and that information can be sent directly to the device 10. FIG. 35 shows a Bluetooth connection between the smartphone and the device 10. However, other connection connection types can be used, such as Wi-Fi.

FIG. 36 shows one possible communication scenario, comprising: a Bluetooth beacon 41 that sends out a signal denoting a specific location within a room, building, city, stadium, recreation park or other location that would benefit from such a device installation; and a person or persons 2, wearing the device 10. The device 10 can pick up this location transmission and store it in its internal memory. A smartphone 43 is synchronised with the device 10. The smartphone can also receive the same location transmission and the two signals together can be used to give a more accurate reading. The smartphone 43 may transmit information over a network 44 such the internet, to any service that is authorised to use the information that can be transmitted. This service may include but is not limited to service application, social networks, databases, websites, non-public systems, medical applications and military applications. A second smartphone 45 can receive data from the first smartphone 43 and may be used for connected applications between devices, the cloud or other connection type known in the art.

### Application Embodiments

Real-Time and Non-Real-Time Medical Patient Monitoring

An API (Application Programming Interface) can be used by medical organisations to develop many different patient monitoring applications that may or may not include the device 10 being attached to the patient. Because the device 10 contains electronics that are able to sense pressure and movement, it is possible to attach a number of the devices 10 to the mattress, bedclothes, bed or clothing as well as the patient's body, to monitor all types of activities. Using the device 10 connected wirelessly (e.g. via Bluetooth) to other sensors, such as stretch and bend sensors, will enable the monitoring of all kinds of behaviours. All or some of the devices 10 can be grouped together to give a customisable data collection. Although standard single device monitoring is possible, the grouped device monitoring functions give much more flexibility, both to the monitoring of activities and the application design.

Real-Time and Non-Real-Time Military and Rescue Personnel Monitoring

There are many situations where active military personnel are in situations that place their bodies under extreme stress. The easy application of the device 10 and its associated stretch and bend sensors, coupled together with its disposability, robustness in extreme conditions, ability to store the data locally, ability to be grouped together to monitor multiple subjects, such as a team and its encrypted data function, makes this device 10 extremely compatible with operational personnel.

### Alternative Embodiments

The above embodiments and sequence of events and the accompanying drawings are intended to be illustrative only and depict only some possible embodiments; however, other embodiments that become apparent upon reading the description and drawings may also fall within the scope of the invention, as defined by the accompanying claims.

1. A biometric sensor device for attachment to the skin of a user, comprising:
a. an electronic device arranged to receive biometric signals or data from the user; and
b. a power cell connected or connectable to the electronic device for providing power thereto;
wherein the electronic device and the power cell are provided as discrete, thin, flexible parts connectable or connected together.

2. The biometric sensor device of claim 1, wherein the electronic device and power cell have respective power contacts for providing power to the electronic device when the power contacts are in mutual electrical contact.

3. The biometric sensor device of claim 2, wherein the power cell includes an electronic control for controlling the supply of power to the electronic device.

4. The biometric sensor device of claim 1, wherein the power cell includes at least one sensor pad for contacting the skin of the user.

5. The biometric sensor device of claim 1, wherein the power cell has a water-resistant edge for contacting the user's skin.

6. The biometric sensor device of claim 4, wherein electronic device and power cell have respective data or signal contacts for providing biometric data or signals to the electronic device when the data or signal contacts are in mutual contact.

7. The biometric sensor device of claim 4, wherein the at least one sensor pad is provided on an inner face of the power cell, arranged to be placed in contact with the user's skin, and the electronic device is attached or attachable to an outer face of the power cell.

8. The biometric sensor device of claim 7, wherein the electronic device includes an outer edge or rim for adhesion to the power cell.

9. The biometric sensor device of claim 1, wherein the electronic device includes at least one sensor pad for contacting the skin of the user.

10. The biometric sensor device of claim 9, wherein the at least one sensor pad is provided on an inner face of the electronic device, arranged to be placed in contact with the user's skin, and the power cell is attached or attachable to an outer face of the electronic device.

11. The biometric sensor device of claim 1, wherein the electronic device and/or the power cell has one or more adhesive layers for securing the electronic device and the power cell together.

12. The biometric sensor device of claim 11, wherein the adhesive layer(s) include electrically conductive adhesive arranged to secure electrical contacts between the electronic device and the power cell.

13. The biometric sensor device of claim 11, wherein the adhesive layer(s) include hydrophobic adhesive arranged to prevent moisture ingress between the electronic device and the power cell.

14. The biometric sensor device of claim 1, wherein the electronic device and the power cell include alignment indicia enabling alignment of the electronic device and the power cell when attached together.

15. The biometric sensor device of claim 1, wherein the power cell is actuable so as to activate the power cell.

16. The biometric sensor device of claim 15, wherein the power cell comprises a capsule containing electrolytes, the capsule being breakable so as to activate the power cell.

17. The biometric sensor device of claim 16, wherein the capsule is provided between printed parts of the power cell.

18. The biometric sensor device of claim 1, wherein the electronic device includes a processor for processing biometric signals and/or data, and/or for controlling the device.

19. The biometric sensor device of claim 1, wherein the electronic device includes memory for storing biometric signals/data, other data received by the device, configuration data or settings for the device, and/or program code for execution by the electronic device.

20. The biometric sensor device of claim 1, wherein the electronic device includes one or more wireless communication interfaces.

21. The biometric sensor device of claim 1, wherein the electronic device includes geolocation circuitry.

22. The biometric sensor device of claim 1, wherein the electronic device includes an accelerometer.

23. The biometric sensor device of claim 1, wherein the electronic device includes an orientation and/or position sensor.

24. The biometric sensor device of claim 1, wherein the electronic device includes flexible encapsulating material for protecting electronic components of the electronic device.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)
